(19) United States
(12) Patent Application Publication
(10) Pub. No.: US 2012/0151676 A1
(43) Pub. Date: Jun. 21, 2012

(54) Comfey table/ surgery, recovery, transfer, pregnancy

(75) Inventor: Joebina Elizabeth Ellingsen, Effort, PA (US)

(73) Assignee: Joebina Elizabeth Ellingsen, Effort, PA (US)

(21) Appl. No.: 12/927,026

(22) Filed: Dec. 21, 2010

(51) Int. Cl. A61G 13/02; A61G 7/14; A61F 7/00; A47C 21/04

(52) U.S. Cl. 5/600; 5/421

(57) ABSTRACT

The comfey table system is a mental frame with attachment holes for support bars and rods with pins that hold the hydraulic legs, head, arms, chest, hips, knees and back attachments in place on the metal frame there is a control pad and wires that connect to the legs that control side to side and up and down positioning of the tables. All five versions can be lengthened and expanded width wise to accommodate any size patient. The comfey system consists of two surgery table tops, pregnancy, transfer, and recovery tables. The surgeon now can place the patients upside down and any other position and can sit down and perform any surgery of today or tomorrow while also controlling patient's blood flows temps with the heating and cooling pad.

The Comfey Table (surgery) pg. (1)

Top view FIG. (1)
1) Top of segmented table (3″×6″×18″ to 36″) lengths of stainless steel slide in and out of each other allowing table to bend.
2) Neck indentation for various head and shoulder attachments.
3) Positioning holes for attachments which also change the width of the various tables to fit the various sizes of patients.(note that the heating and cooling messaging gel pad can be placed on the tables after proper sizing has been completed.

Side view FIG. (2)

1) Head and shoulder restraint attachment

2) Hole for locking pin that holds hydraulic legs in place (Note that the holes are on the long side of the table thus allowing the table to be lifted only on the long sides of the metal frame work)

3) Segmented table

4) Hydraulic legs

5) Base of hydraulic legs with a roller ball that allows the table to be moved around

Side view (body attachments) FIG. (2a)

1) Face up head and shoulder attachment

2) Left or right (left side shown) body restraint attachment

3) Left or right side (left side shown) head and shoulder attachment

4) Face down head and shoulder attachment

5) Metal anchors with pins to secure attachments (Note all attachments come in petite, extra small, small, medium, medium large, large, extra large and triple extra large sizes.)

6) Foot step top view with metal anchor

Side view (segmented table top) FIG. (3)

1a) metal strips connection pivot point

The Comfey Table FIG. (8) pg (2)

Bottom view of hydraulic foot pad and roller ball

1a) foot pad (base of hydraulic leg)

2a) roller ball

FIG. (9) side view of foot pad and roller

1) Foot pad base

2) Roller ball

Bottom end view FIG. (10)

1) Table shown with leg supports attached on the short sides of table allowing table to be tilting table right to left on the long side instead of head to foot elevating

2) Hydraulic leg

3) Rounded hydraulic leg connected at feet for side to side tilting

The Comfey Table pg (3)

Side view FIG. (11)

1) Table top

2)Support connection (supporting metal bars)for top of pillar

3) Hydraulic leg

4) Hydraulic leg pillars attached for side to side tilting

5) Holes for hydraulic leg connection to support bars

Bottom view of surgery table FIG. (12)

1) Bottom view of table

2) Connection point for electric feed to hydraulic leg

3) Metal support frame

4) Metal support bars attached to the metal frame

5) Hydraulic legs positioning spots on metal frame for segmented table version

6) Locking pin for hydraulic legs

The Comfey Table

Recovery table pg (4)

Top view FIG. (13)

1) Table elevated to vertical position

2) Foot step

3) Hydraulic leg raised to 24″

4) Hydraulic leg foot base and roller

5) Hydraulic leg connected on the long side for vertical lift and decent

6) Handle grasp for security while table is in motion

7) Shows table leg at 3″ start position to 2′ table laying down position

Recovery table pg (4)

Top view FIG. (14)

1) Table top

2) Foot step

3) Knee supports

4) Hanger for sling anchored to table

5) Hand grasp

6) Suspended arm sling

7) Knee attachment bend point

Side view FIG. (15) pg (4)

1) Table side view

2) Foot step

3) Knee support up

4) Knee support down

5) Hand grasp

6) Suspended arm sling elevated (6a) arm sling down

7) Hydraulic leg

8) Anchor pin for arm sling and positioning hole

9) Suspended leg sling elevated (leg can also hang down)

10) Leg sling

Top view leg attachments FIG. (16)

3) Full support for knee 3a) open knee support in down position

The Comfey Table for pregnancy pg (5)

FIG. (17)

Side view

1) Head attachment

2) Chest support attachment(shoulder and ribs)

3) Hip and pelvis support attachment

4) Arm support attachment

5) Padded hip and leg full area attachment

6) Hydraulic legs supports extended to maximum height 30″

7) Hip and pelvis attachment support bar

8) Chest (Shoulder and rib) support bar(near abdomen)

9) Chest (shoulder and rib)support bar (near neck)

10) Head support

11) Metal bed frame work system

12) Holes for various body attachments to be connected to and placement rods and support bars

Top view FIG. (18)

1a) head attachment

2a) chest attachment

3a) hip and pelvic attachment

4a) arm support attachment

5a) leg support attachment

6a) threaded placement rods adjusts width of supports for the hip and pelvic adjustments and are adjusted by turning the handles

7a) threaded placement and positioning rods adjust the length of chest and hip attachments

8a) holes for adjustments on head, shoulder, arms, chest ribs, hips, pelvis, and knee support attachments

9a) shoulder, chest, and rib placement support bars

10a) head placement and support rods

11a)locking pins to secure placement and support rods

12a) arm support attachment and placement rods

The comfey Table pregnancy pg (5)

Continued from FIG. (18)

13a) computerized electric box for hydraulic legs and up and down height adjustments

14a) plug for computerized control pad for hydraulic legs

15a) control pad for each hydraulic leg

16a) metal support system

The comfey Table pregnancy table pg (6)

Bottom view FIG. (19)

1) Metal support system for attachments on table

2) Head attachment support bars

3) Head support attachment

4) Shoulder and chest and rib support attachment

5) Shoulder and chest and rib support bars

6) Hip and pelvic support attachment

7) Hip and pelvic support bars (all support bars are 10″×2″×3″

8) Locking pins to secure placement and support bars

9) Threaded placement and position handled rods for hips and pelvic supports (for placement supports and adjustments of the width of the hips and pelvis)

10) Hydraulic legs attaching placements

11) Threaded placement and position handled rods for adjusting length for position of hips and pelvis supports attachments

12) Computerized electrical control box for hydraulic legs

13) Plug for computerized control box

14) Control pad for each hydraulic leg

15) Head placement and support rods

16) Shoulder chest and rib support attachment placement and support rods

17) Arm support placement and support rods

18) Arm support attachments

19) Leg section attachment holes and pins

20) Leg section support bars

21) Leg attachment

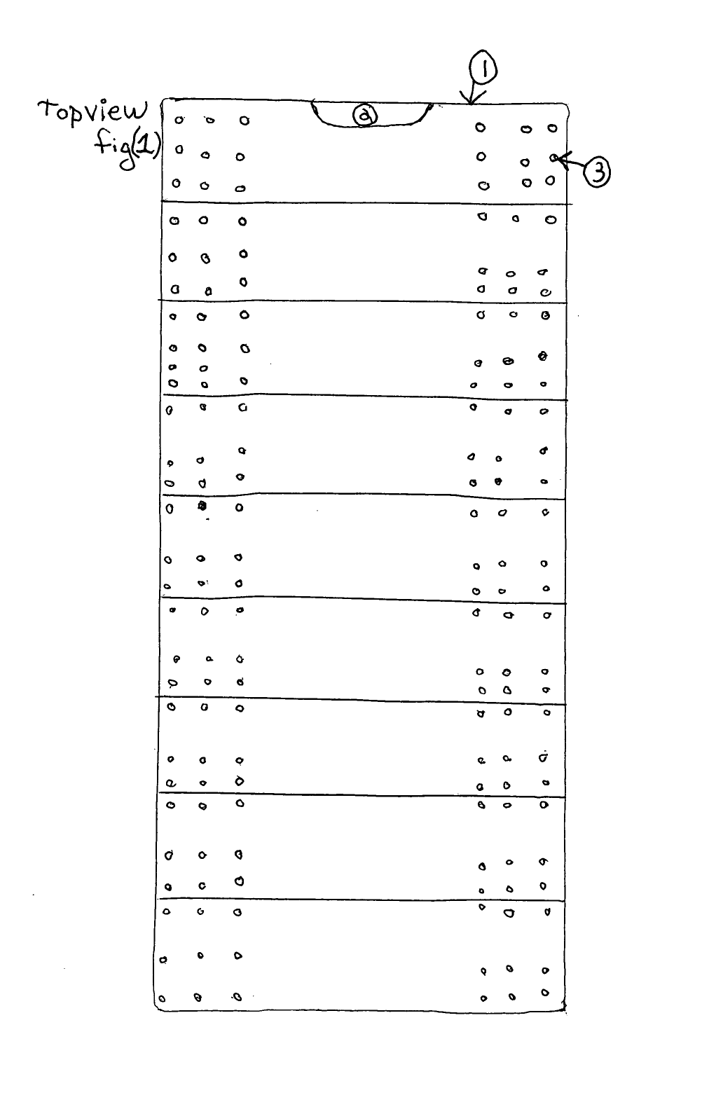

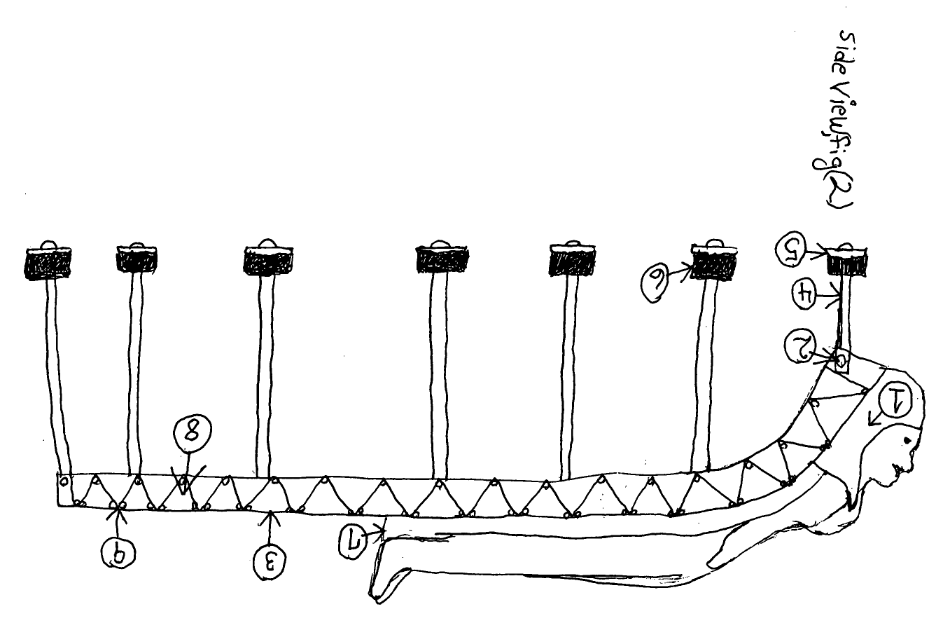

The Comfey table/heating and cooling messaging gel pad For recovery, surgery, pregnancy tables pg (7) FIG. 20)

1) 3′×8′ heating and cooling messaging gel pad with face hole up position shown. Used for surgery preparation because it increases or decreases head temperature, helping the surgery that's being performed

2) Face hole to fit patient

3) 1″ tubes channel heated or cooled water through the messaging gel pads

4) Heating box that water passes through heated by an heating element and then is pumped through the messaging gel pads

5) 1″ tube between the heater and cooling boxes

6) Electric supply between heating and cooling boxes

7) Cooling box unit with pump to force cooled water through the messaging gel pads

8) Plug and electric supply to control pad and the heating and cooling boxes with pumps

9) Control pad for hot and cold water supply that flows throughout the gel pad

10) Electric supply between control pad and heating and cooling boxes

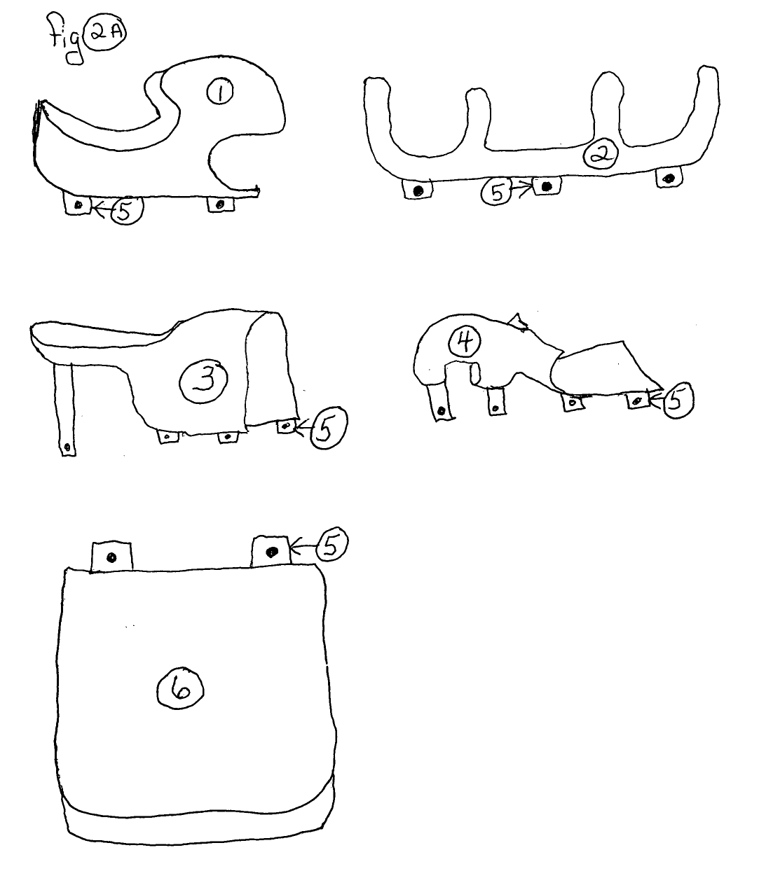

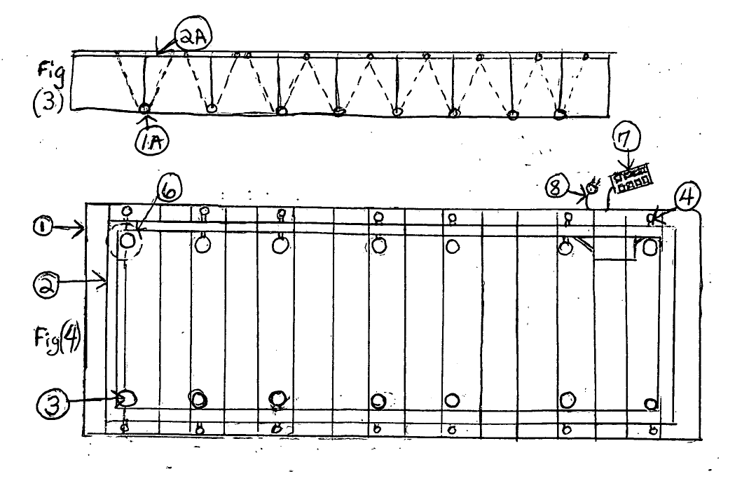

(FIG. 21)

11) Full hot and cold pad for the back of the patient 3′×8′

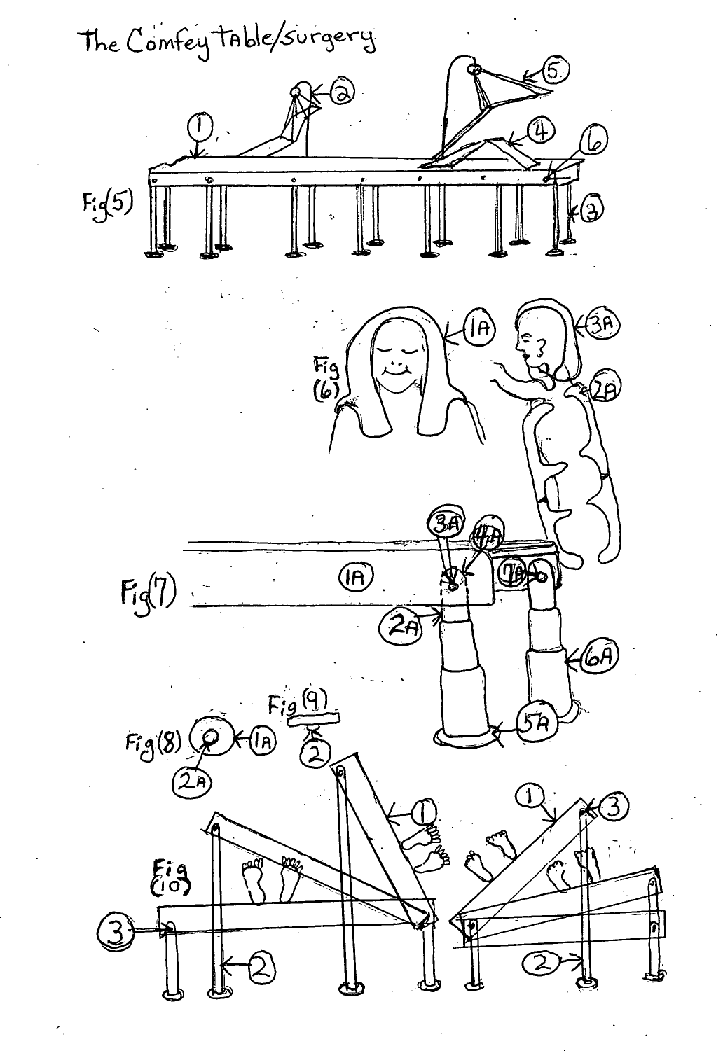

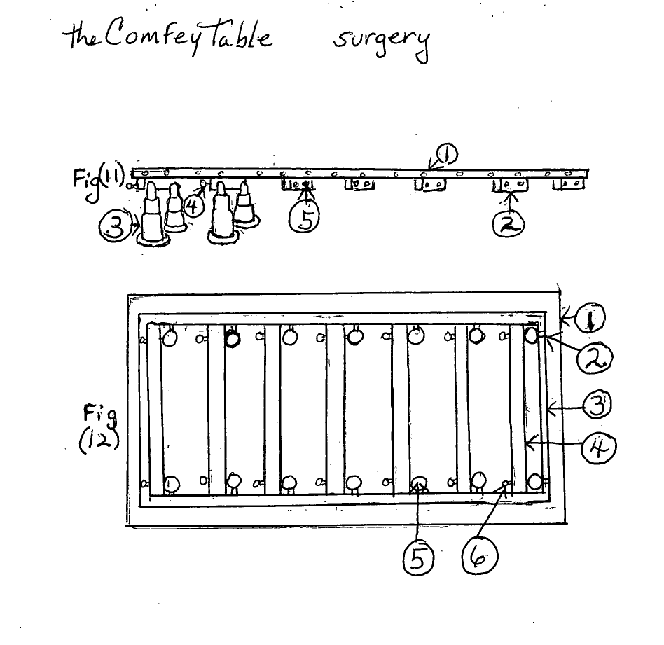

(FIG. 22)

12) Head hot and cold gel pad 24″×24″ with face hole for comfort

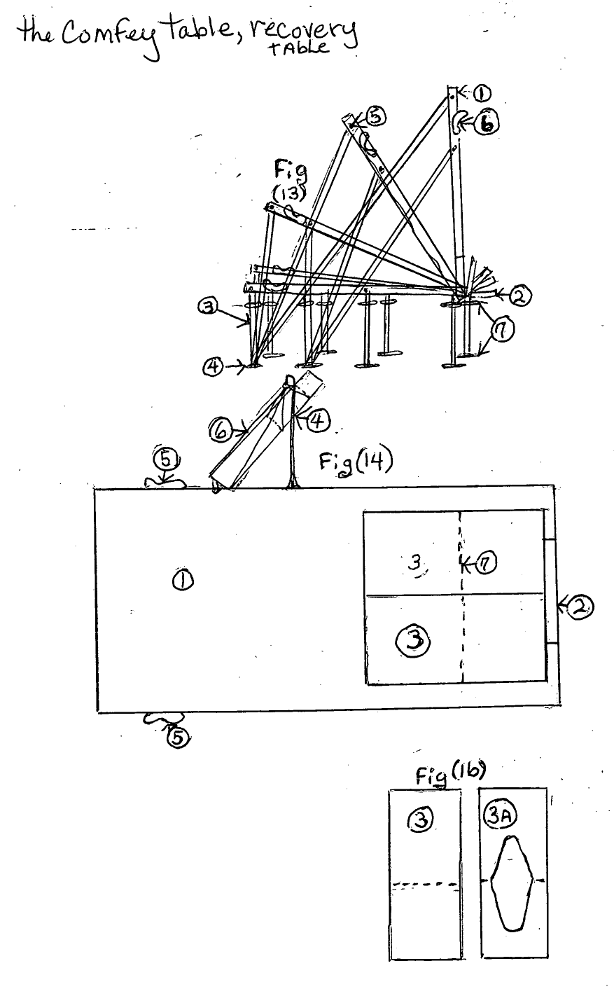

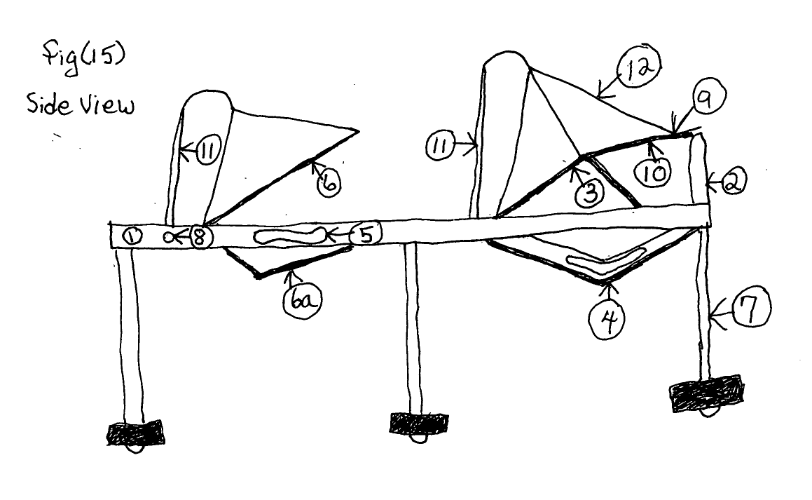

(FIG. 23)

13) Hot and cold gel pad 3′×4′ for upper or lower body

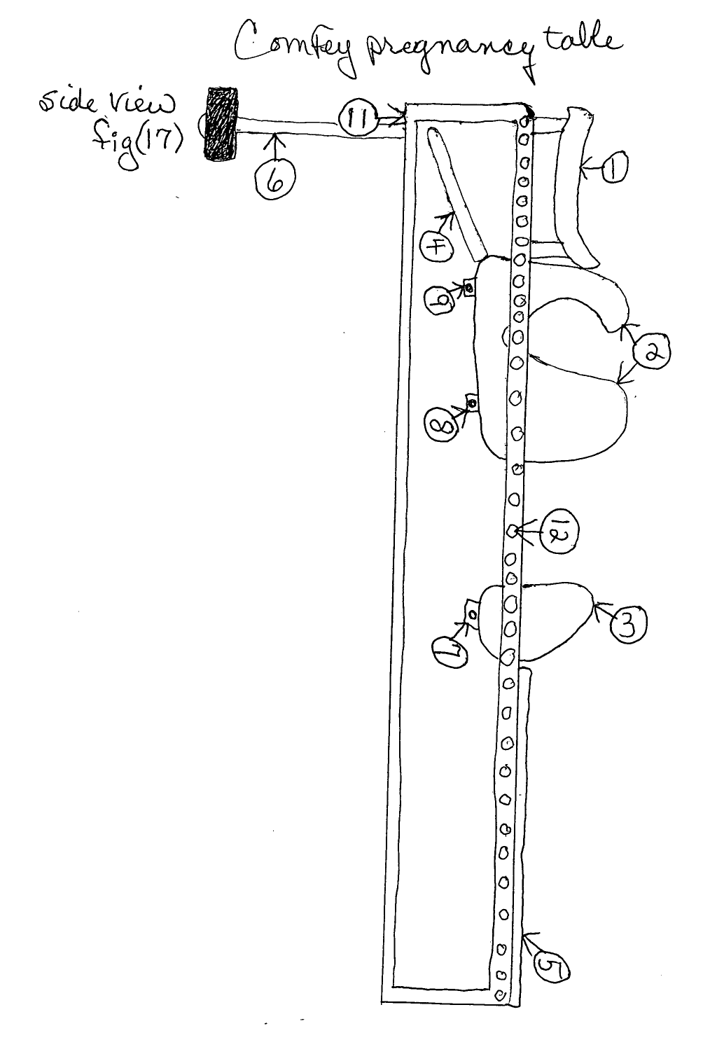

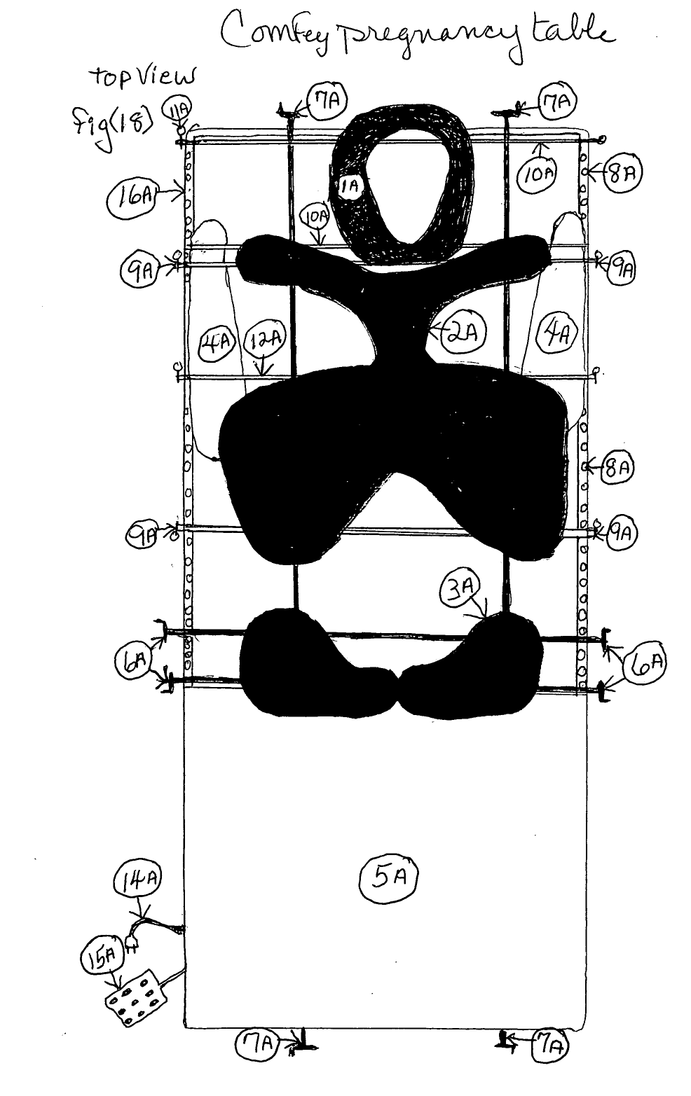

(FIG. 24)

14) 2′×4′ arm or leg hot and cold gel pad

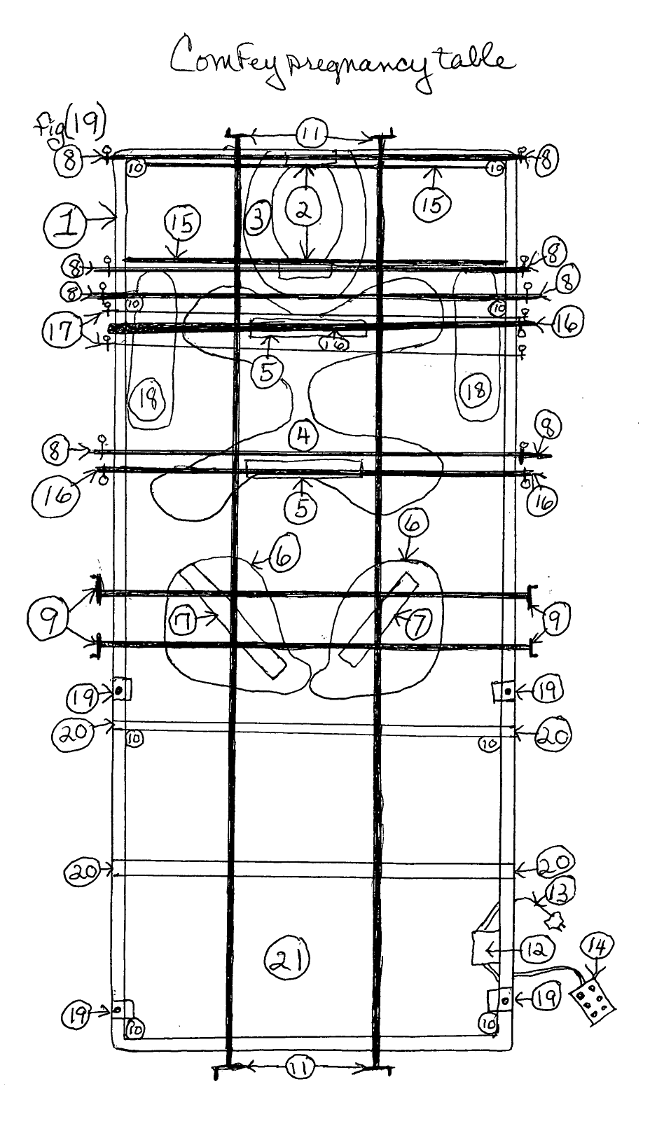

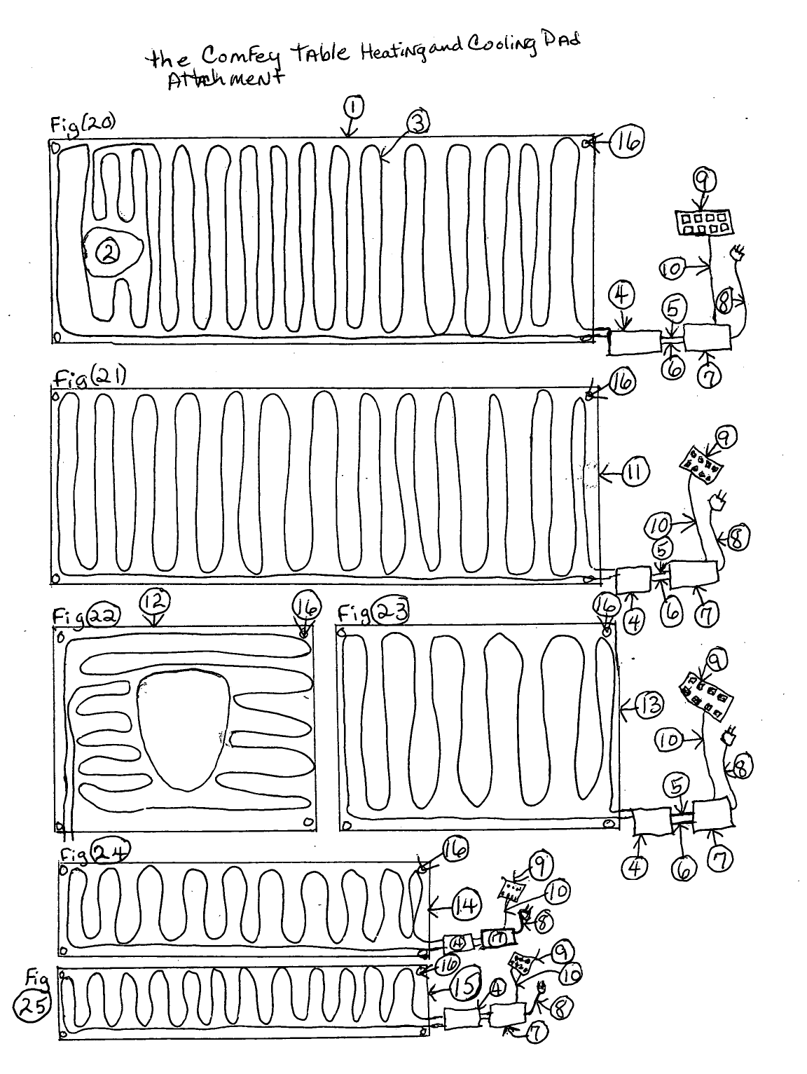

(FIG. 25)

15) 2′×4′ armor leg gel pad

(FIGS. 20 to 25)

16) snaps that hold hot and cold messaging gel pads in place

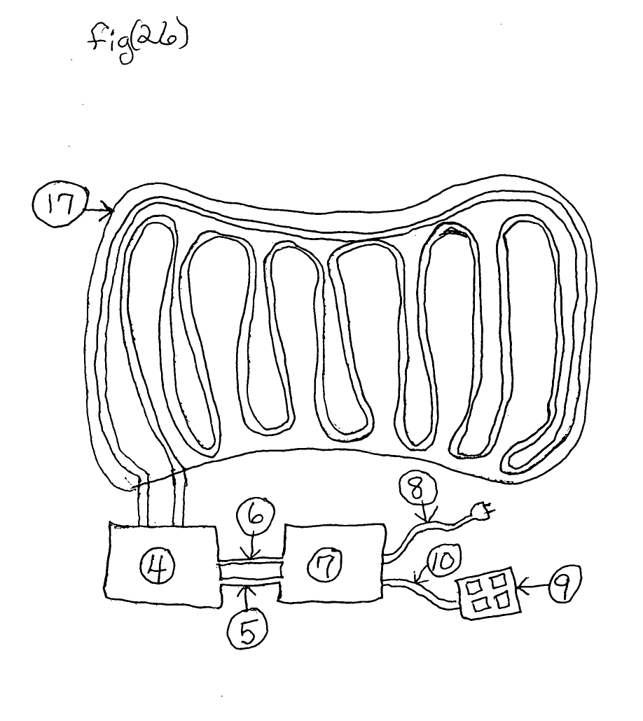

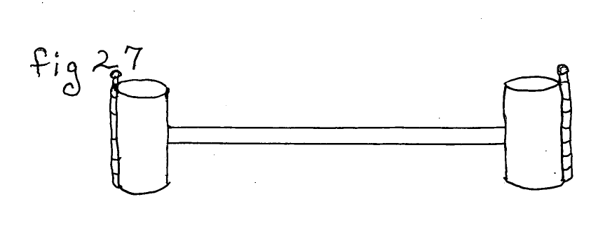

(FIG. 26) (17) breast heating and cooling pad (note no snaps just lays on the breast)

FIG. (2) from pg (1)

6)lead weights for hydraulic legs

7) hot and cold gel pad

8) triangular shaped edge of segmented table top allows the necessary freedom for movement

9) circular crimps with steel c shaped rods hold segmented top together

FIG. (15) from pg (4)

11) leg or arm sling hanger

12) cable used to suspend leg or arm slings

FIGS. (20-26) (4 thru 10 are the same specifications from FIGS. (20 to 26) and FIG. (20-26) are different sizes of the same heating and cooling pads for the surgical, recovery, transfer, and pregnancy tables.

1. That the comfey table system comprising of five different tables is a framework with attachments that convert into each version of the five tables, further comprising, of two types of surgical tables, and a pregnancy, transfer, and recovery table, Further comprising, of different sized heated or cooled gel pads. And limited to each table being used only for what it is designed to do any other use could cause injury
